(19) United States
(12) Patent Application Publication
(10) Pub. No.: US 2006/0286573 A1
(43) Pub. Date: Dec. 21, 2006

(54) Method for determining nucleotide sequence of nucleic acid molecule by utilizing scanning probe microscope

(75) Inventor: Kazuhiro Takada, Sagamihara-shi (JP)

Correspondence Address: FITZPATRICK CELLA HARPER & SCINTO, 30 ROCKEFELLER PLAZA, NEW YORK, NY, 10112, US

(73) Assignee: Canon Kabushiki Kaisha, Tokyo (JP)

(21) Appl. No.: 11/386,725

(22) Filed: Mar. 23, 2006

(30) Foreign Application Priority Data
Mar. 25, 2005 (JP) 2005-088618

(51) Int. Cl. C12Q 1/68

(52) U.S. Cl. 435/6

(57) ABSTRACT

A nucleotide sequence of a single-stranded nucleic acid molecule immobilized on a substrate is determined with an SPM by analyzing a shape of bases constituting the single-stranded nucleic acid molecule by scanning over a nucleic acid chain of the single-stranded nucleic acid molecule immobilized on the substrate with a probe provided to the SPM; preparing a standard nucleic acid-binding probe and detecting the presence or absence of interaction between the bases constituting the single-stranded nucleic acid molecule and the standard nucleic acid by scanning over the nucleic acid chain of the single-stranded nucleic acid molecule using the standard nucleic acid-binding probe; and determining the nucleotide sequence of the nucleic acid chain by identifying the kinds of bases existing in the nucleic acid chain constituting the single-stranded nucleic acid molecule from the results obtained above.

### BACKGROUND OF THE INVENTION

1. Field of the Invention

The present invention relates to methods for determining nucleotide sequences of nucleic acid molecules. More specifically, the present invention relates to methods for determining nucleotide sequences of single-stranded nucleic acid molecules, such as DNA and RNA, by utilizing a scanning probe microscope.

2. Description of the Related Art

As typical methods for determining nucleotide sequences of DNA, the Maxam-Gilbert method which is a chemical technique and the Sanger method which is an enzymatic technique are known.

In the Maxam-Gilbert method, an end of a single-stranded DNA of interest is labeled with a labeling agent; the DNA chain is cut by a base-specific chemical reaction into cleavage products; and the cleavage products are isolated by electrophoresis. Then, the base lengths of the cleavage products having the labeling agent at the end are measured; and information for the entire nucleotide sequence of the DNA is restructured on the basis of information of the bases which were cut at the cleavage points. However, since the amount of each cleavage product is small, the sensitivity is low. Additionally, harmful chemical reagents are used in a series of chemical treatments. Consequently, the method is rarely used nowadays.

In the Sanger method, a single-stranded DNA of interest is used as a template; and DNA fragments of various lengths are synthesized by extension of a primer at the 3′-end by utilizing repair synthesis of a DNA polymerase. The 3′-ends of the extended fragments having various lengths are terminated by bases labeled so as to be specific to the respective bases. The fragments are isolated by electrophoresis and each base length of the fragments is determined. At the same time, the base at the 3′-end is identified by utilizing the labeling which is specific to the respective bases. The nucleotide sequence of the extended DNA chain at the 3′-end of the primer is restructured on the basis of the information for the base length and the kind of the base of the 3′-end of each fragment. Namely, the nucleotide sequence of the template, i.e., the single-stranded DNA of interest, is determined by determining the nucleotide sequence of amplified products having a sequence complementary to the single-stranded DNA used as the template. When a sequence of interest is RNA, generally, cDNA having a nucleotide sequence complementary to the RNA is previously synthesized using a reverse transcriptase by using the RNA as a template. Then, the nucleotide sequence of the cDNA is determined using the cDNA as a template as in the above-mentioned method. The nucleotide sequence of the RNA is determined on the basis of the nucleotide sequence of the cDNA which is complementary to the RNA.

Since the Sanger method utilizes amplified products of a DNA polymerase using a single-stranded DNA of interest as a template, its detection sensitivity is generally higher than that of the Maxam-Gilbert method. Therefore, it is advantageous that the amount of a single-stranded DNA itself (template) to be measured can be small. Because of this advantage, the Sanger method is most widely utilized for sequencing RNA and DNA, at present.

Each of the above-mentioned two methods is an analytical method which is a combination of a chemical or enzymatic reaction and an analysis of length of nucleotide fragments isolated by electrophoresis. As an analytical method different from such a method, a scanning probe microscope (SPM) represented by a scanning tunnel microscope (STM) and an atomic force microscope (AFM) is utilized.

By using the STM, two-dimensional information in the atomic scale with respect to the surface profile of a sample can be obtained by scanning the sample two-dimensionally in relation to a probe while measuring tunnel current flowing between the sample and the probe.

By using the AFM, two-dimensional information with respect to the surface profile of a sample can be obtained by scanning the sample two-dimensionally in relation to a probe while measuring atomic force between the sample and the probe.

DNA is composed of four bases: adenine (A), thymine (T), guanine (G), and cytosine (C). Each base has a molecular shape different from that of each other. Accordingly, the structural shape of the base portion is previously confirmed by measuring it by the SPM technology. Two-dimensional information of a single-stranded DNA molecule in the atomic scale is obtained by measuring structural shapes of bases of the DNA molecule along the nucleic acid chain by the SPM technology. The kind of the base can be determined by comparing the two-dimensional information with the previously confirmed structural shape of each base portion. Therefore, information about the nucleotide sequence of the single-stranded DNA molecule can be successively obtained on the basis of the results of the measurement along the nucleic acid chain.

Each base constituting a DNA molecule has a characteristic to form a base pair between complementary bases, i.e., A-T and G-C, in an aqueous solution. A method utilizing this characteristic is disclosed in Japanese Patent No. 3368934. A base sequence is determined by forming a base pair in an aqueous solution and comparing the shape of the DNA molecule to which the base pair has been formed and the shape of the original DNA molecule by using the SPM technology. Specifically, a predetermined base is supplied to a single-stranded DNA molecule to form a base pair; and the position where the base pair is formed in the single-stranded DNA molecule is determined by observing and comparing the shape of the DNA molecule to which the base pair is formed and the shape of the original DNA molecule by the SPM technology. This process is repeated for four bases: adenine (A), thymine (T), guanine (G), and cytosine (C). As a result, the positions of thymine (T), adenine (A), cytosine (C), and guanine (G), which are complementary to the above-mentioned bases, respectively, can be identified on the single-stranded DNA of interest. The nucleotide sequence of the DNA can be determined by aggregating the information.

The above-mentioned Maxam-Gilbert method and Sanger method are analytical methods which combine a chemical or enzymatic reaction and analysis of length of nucleotide fragments isolated by electrophoresis. In these methods, nucleic acid fragments prepared by the chemical or enzymatic reaction are measured instead of a DNA or RNA molecule itself in a sample. Namely, these methods are analytical methods for obtaining information necessary for indirectly determining a nucleotide sequence by using characteristics of the nucleotide sequence of a nucleic acid molecule. In these methods, the nucleotide sequence is not determined by directly observing a DNA or RNA molecule itself in a sample. Furthermore, a certain amount of nucleic acid is necessary for achieving sensitivity sufficient for measuring the nucleic acid fragments prepared by the chemical or enzymatic reaction. Therefore, when the amount of nucleic acid in a sample is small, amplification of the nucleic acid by a polymerase chain reaction (PCR) method using the nucleic acid as a template is necessary. In general, measurement methodology including a pretreatment process for preparing an amplification product (secondary sample) by the PCR method is employed. When the amplification product is prepared by the PCR method, a primer for the PCR is further necessary. Furthermore, partial information of the nucleotide sequence for designing a nucleotide sequence of the primer for the PCR is also necessary.

On the other hand, in the analytical method of a nucleotide sequence using the SPM technology, the nucleotide sequence is directly determined by determining the kinds of bases constituting the nucleic acid chain by measuring a DNA molecule itself as a measurement target. The STM method itself has a high resolution so that positions of the individual atoms can be identified and molecular structure of the bases can be determined. However, in order to apply the STM method to a sample, the sample must be electrically conductive. Therefore, the STM method is not necessarily adequate for observing DNA. On the other hand, the AFM can be applied to the observation of a nonconductive sample, but the resolution is inferior to that of the STM. Therefore, it is difficult to identify kinds of the respective bases constituting a nucleic acid chain by the observation with the AFM alone.

In the method disclosed in Japanese Patent No. 3368934, base pairs are formed by immersing a single-stranded DNA sample into an aqueous solution and adding one kind of base which is specific to one kind of base among four bases constituting the nucleic acid chain; and a change in the shape of the base portions where the bases are specifically bound to are observed by utilizing the SPM technology. Namely, the nucleotide sequence of the entire nucleic acid chain is determined on the basis of the information for the positions where the one kind of base bound to form the base pairs. The positions of base portions where base pairs are formed are measured for the respective four kinds of bases: A, T, C, and G. In order to determine the entire nucleotide sequence of DNA of interest, it is necessary to prepare a plurality of DNA fragments complementary to a partial sequence of the entire nucleotide sequence and to form the respective base pairs. In other words, it is necessary to prepare, in advance, secondary samples of DNA fragments having the same nucleotide sequence by utilizing the PCR amplification reaction. Additionally, the formation of the base pair with one kind of base is conducted in a solution. In this view, this method cannot be defined as a method for directly determining a nucleotide sequence by using a DNA sample.

Therefore, a method for directly determining a nucleotide sequence by using an extracted DNA sample is desired.

### SUMMARY OF THE INVENTION

The present invention provides a method that can directly determine a nucleotide sequence of a basically single nucleic acid molecule, without the amplification of the nucleic acid chain, with a high accuracy.

The method for determining a nucleotide sequence of a nucleic acid molecule provided by the present invention is a method for determining a nucleotide sequence of a single-stranded nucleic acid molecule immobilized on a substrate by using a scanning probe microscope. The method includes the steps of:
(I) analyzing a shape of bases constituting the single-stranded nucleic acid molecule by scanning over a nucleic acid chain of the single-stranded nucleic acid molecule immobilized on the substrate with a probe provided to the scanning probe microscope;
(II) preparing a standard nucleic acid-binding probe and detecting the presence or absence of interaction between the bases constituting the single-stranded nucleic acid molecule and the standard nucleic acid by scanning over the nucleic acid chain of the single-stranded nucleic acid molecule using the standard nucleic acid-binding probe; and
(III) determining the nucleotide sequence of the nucleic acid chain by identifying the kinds of bases existing in the nucleic acid chain constituting the single-stranded nucleic acid molecule on the basis of the results obtained in the steps (I) and (II).

In the method according to the present invention, a standard nucleic acid-binding probe is used, and the presence or absence of interaction between bases constituting a single-stranded nucleic acid molecule and the standard nucleic acid is detected by scanning over the nucleic acid chain of the single-stranded nucleic acid molecule. Thus, it is possible to directly determine a nucleotide sequence of a basically single nucleic acid molecule with a high accuracy by using an extracted nucleic acid molecule sample as it is.

Further features of the present invention will become apparent from the following description of exemplary embodiments with reference to the attached drawings.

### BRIEF DESCRIPTION OF THE DRAWINGS

FIGS. 1A and 1B are schematic diagrams respectively showing a step of the method according to the present invention for determining a nucleotide sequence of nucleic acid.

FIG. 2 is a schematic diagram showing a process for analyzing shapes of the bases constituting a nucleic acid molecule by scanning over a single-stranded nucleic acid molecule immobilized on a substrate with a probe provided to a scanning probe microscope.

### DESCRIPTION OF THE EMBODIMENTS

In the method for determining nucleotide sequence of a nucleic acid molecule according to the present invention, a nucleotide sequence of a single-stranded nucleic acid molecule immobilized on a substrate is determined by using a scanning probe microscope. The method includes the steps of:

(I) analyzing a shape of bases constituting the single-stranded nucleic acid molecule by scanning over a nucleic acid chain of the single-stranded nucleic acid molecule immobilized on the substrate with a probe provided to the scanning probe microscope;

(II) preparing a standard nucleic acid-binding probe and detecting the presence or absence of interaction between the bases constituting the single-stranded nucleic acid molecule and the standard nucleic acid by scanning over the nucleic acid chain of the single-stranded nucleic acid molecule with the standard nucleic acid-binding probe; and

(III) determining the nucleotide sequence of the nucleic acid chain by identifying the kinds of bases existing in the nucleic acid chain of the single-stranded nucleic acid molecule on the basis of the results obtained in the steps (I) and (II).

In the present invention, the standard nucleic acid-binding probe may be formed by using a single-stranded DNA formed of only one base of adenine, thymine, guanine, or cytosine as the standard nucleic acid.

Additionally, four kinds of single-stranded DNA can be also used as the standard nucleic acid.

In the standard nucleic acid-binding probe used in the step (II), the above-mentioned standard nucleic acid is bound to the surface of the front end of the probe. The probe may be made of the same material and have the same shape as those of the probe used in the step (I).

The detection of the presence or absence of interaction may be conducted, for example, by detecting displacement of a cantilever provided with the probe.

The scanning probe microscope (SPM) may be an atomic force microscope (ATM).

The method for determining a nucleotide sequence of nucleic acid according to the present invention utilizes a phenomenon that interaction is caused between two kinds of bases being complementary to each other, i.e., being able to form a base pair, A-T or C-G, when the two kinds of bases come close to each other in gas phase. In order to detect the interaction arising between the bases of the base pair for every base existing in the nucleic acid chain, a probe method is utilized in which the position resolution is the atomic scale in the SPM. Furthermore, n-mer DNA fragments, An, Tn, Cn, and Gn, which are formed of n bases of one kind of the four bases, A, T, C, and G, respectively, are utilized, for example. Each of these fragments is bound to the front end of the probe and can become close to the base of the nucleic acid chain with high position selectivity. By two-dimensionally scanning along the nucleic acid chain constituting the single-stranded DNA immobilized on a substrate with this probe having the n-mer DNA fragment at the front end, the presence or absence of interaction between each base of the nucleic acid chain and the n-mer DNA fragment formed of one kind of the complementary bases can be detected.

The interaction causes displacement of the probe at the same level as a change in atomic force. Therefore, such displacement can be detected by utilizing a detection system of the SPM. In the present invention, each base of the nucleic acid chain constituting the single-stranded DNA molecule can be finally identified by aggregating the results measured for the presence or absence of interaction by using four types of probes of which front ends are bound with An, Tn, Cn, or Gn.

For conducting the present invention, an SPM may be used to analyze shapes of bases of a single-stranded nucleic acid molecule immobilized on a substrate by using a probe and to obtain information about the position of each base as information capable of creating a two-dimensional image.

Furthermore, the SPM may be used in analysis using a standard nucleic acid-binding probe for analyzing the presence or absence of interaction between each base of a single-stranded nucleic acid molecule and the standard nucleic acid by using the standard nucleic acid-binding probe and for obtaining the information about the presence or absence of the interaction at the position of each base with the standard nucleic acid as information capable of creating a two-dimensional image.

Additionally, the SPM may have a mechanism for identifying the kind of each base of the single-stranded nucleic acid molecule on the basis of the information obtained above and converting it into one-dimensional information showing a nucleotide sequence of the nucleic acid chain.

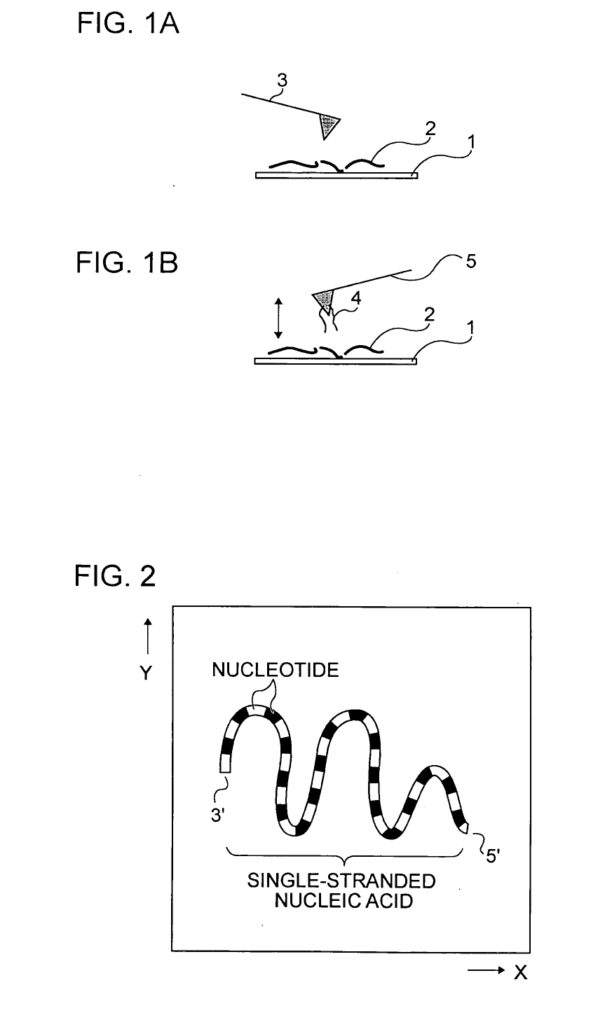

In the step (I) of the present invention, the SPM is used, and the shape of a single-stranded nucleic acid immobilized on a substrate, in particular, the shape of each base, is measured by using a probe, FIG. 1A schematically shows this step. As shown in FIG. 1A, the shape of each base of a single-stranded nucleic acid 2 is measured using a probe 3. With this step, the position information for each base on the nucleic acid chain can be two-dimensionally obtained. As shown in FIG. 2, a single-stranded nucleic acid is immobilized on a substrate having an X-Y plane, and the position and shape of each base are measured by scanning over this single-stranded nucleic acid with a probe.

The nucleic acid to be measured may be any nucleic acid molecule such as DNA, RNA, cDNA, and genome DNA as long as it is immobilized on the substrate as a single-stranded nucleic acid molecule. Since the SPM is used for measuring the single-stranded nucleic acid immobilized on the substrate, a substrate having a surface with high planarity is used. Examples of such a substrate include cleaved graphite, cleaved mica, a substrate of cleaved mica having an evaporated gold film on the surface, and a silicon substrate.

The single-stranded nucleic acid molecule may be immobilized on the surface of the substrate by any method as long as the structure of the single-stranded nucleic acid is not changed. Generally, the single-stranded nucleic acid molecule is immobilized by dropping a solution containing a predetermined concentration of the single-stranded nucleic acid onto a substrate and evaporating the solvent so that the single-stranded nucleic acid is fixed on the substrate surface; or immersing the substrate into a solution containing a predetermined concentration of the single-stranded nucleic acid so that the single-stranded nucleic acid is adsorbed on the substrate surface.

When the shape of the single-stranded nucleic acid immobilized on the substrate surface is analyzed by using the SPM, an AFM is generally used as the SPM for measuring the shape because the single-stranded nucleic acid itself does not have electrical conductivity. There are several modes in the AFM such as a contact mode and a non-contact mode including an intermittent contact mode and a cyclic contact mode (e.g., Tapping Mode: a registered trademark of Digital Instrument). Any mode may be used as long as the AFM can observe the shape of the single-stranded nucleic acid along the nucleic acid chain and identify the base positions. In this step, common probes such as a common silicon probe, a silicon-nitride probe, and a carbon nano-tube may be used. When the substrate is electrically conductive, the single-stranded nucleic acid molecule immobilized on the substrate can be observed by a scanning tunnel microscope.

Furthermore, SPM-derived technology other than a method directly observing the shape can be applied when such technology can two-dimensionally measure the shape, i.e., the position of each base, of the single-stranded nucleic acid immobilized on the substrate surface.

In the step (II), a standard nucleic acid-binding probe is brought close to a position of each base constituting the single-stranded nucleic acid, and the presence or absence of interaction between the base and the standard nucleic acid is measured. FIG. 1B schematically shows this step. As shown in FIG. 1B, the presence or absence of interaction between the bases constituting the single-stranded nucleic acid molecule 2 immobilized on the substrate 1 and the standard nucleic acid 4 is detected using the standard nucleic acid-binding probe 5.

As the standard nucleic acid, for example, n-mer DNA fragments, An, Tn, Cn, and Gn, which are formed of n bases of one kind of the four bases, A, T, C, and G, respectively, can be utilized.

In the step for measuring the presence or absence of interaction between a base and standard nucleic acid, the accuracy of position selectivity is needed to be equivalent to that in the step (I). Consequently, the standard nucleic acid-binding probe is required to have acuity at least equivalent to that of the front end of the probe used in the AFM. Therefore, the material and the shape of the probe used in preparation for the “standard nucleic acid-binding probe” may be the same as those of the probe used in the step (I). The front end of the probe may be a sharper shape.

The presence or absence of interaction between a base and standard nucleic acid can be measured by the AFM by detecting displacement of the probe position by a change in the atomic force. A typical method is a force curve technique which is a detection system utilized in the AFM too. Namely, the back surface of the probe is irradiated with laser light and the reflected light is detected by a light detector such as a photodiode. The displacement of a cantilever provided with a probe changes a reflection angle of the laser light irradiated to the back surface. The cantilever is adjusted such that the reflected laser light equally enters each segment of the light detector, in general, a segmented photodiode (generally, having two segments), when the cantilever is not displaced. On the other hand, when the cantilever is displaced, the reflected laser light enters each of the segments with bias. Therefore, the interaction can be detected as a difference in output of each segment of the photodiode.

The front end of the “standard nucleic acid-binding probe” is brought close to the position of each of the identified bases. The occurrence of the interaction causes displacement of the cantilever. Thus, the displacement is detected and the presence or absence of the interaction is analyzed.

For example, n-mer DNA fragments, An, Tn, Cn, and Gn, which are formed of n bases of one kind of the four bases, A, T, C, and G, respectively, are used as the standard nucleic acid of the “standard nucleic acid-binding probe”. The presence or absence of interaction with each of the four kinds of n-mer DNA fragments is sequentially analyzed.

In the step (III), the kinds of bases existing in the nucleic acid chain of the single-stranded nucleic acid molecule are identified on the basis of the results obtained in steps (I) and (II) using the following criteria.

By using each of the four kinds of “standard nucleic acid-binding probes” of An, Tn, Cn, and Gn, the presence or absence of interaction with those four kinds of DNA fragments is analyzed, and the results of the analysis are compared. The results of the step (I) for identifying the base at a position are:

(i) when the base at the position is adenine (A), interaction measured by the “standard nucleic acid-binding probe” having Tn as the standard nucleic acid, i.e., interaction corresponding to the formation of A-T base pair, is specifically detected;

(ii) when the base at the position is thymine (T), interaction measured by the “standard nucleic acid-binding probe” having An as the standard nucleic acid, i.e., interaction corresponding to the formation of T-A base pair, is specifically detected;

(iii) when the base at the position is guanine (G), interaction measured by the “standard nucleic acid-binding probe” having Cn as the standard nucleic acid, i.e., interaction corresponding to the formation of G-C base pair, is specifically detected; and

(iv) when the base at the position is cytosine (C), interaction measured by the “standard nucleic acid-binding probe” having Gn as the standard nucleic acid, i.e., interaction corresponding to the formation of C-G base pair, is specifically detected.

One-dimensional information corresponding to the nucleotide sequence of the nucleic acid chain is obtained by sequentially determining which criteria of the above-mentioned (i) to (iv) is applied to each base identified along the nucleic acid chain.

### Embodiments

The present invention will now be specifically described with reference to Examples. While the exemplary embodiments shown below are examples of the present invention, it is to be understood that the invention is not limited to these exemplary embodiments.

### Embodiment 1

(Preparation of Substrate for Immobilizing DNA for Scanning Probe Microscope Observation)

A clean cleavage face was prepared by cleaving natural mica (Nilaco Corp.) having a 10 mm×10 mm plane into squares parallel to the plane. Then, the debris generated during the cleavage is removed by blowing nitrogen gas. Thus, a mica substrate was prepared.

The thus prepared mica substrate was set in a vacuum vapor-deposition apparatus being able to heat the substrate, and the vapor-deposition chamber was evacuated to about 1×10⁻⁴ Pa. Then, the mica substrate was annealed at 600° C. for about 5 hr. On the surface of the annealed mica substrate, a gold coating layer was formed by a vacuum vapor-deposition method. The thickness of the gold coating layer was controlled to about 10 nm by monitoring the thickness of the vapor-deposited coating with a quartz oscillator film thickness meter which is provided to the vacuum vapor-deposition apparatus. Continuously, the mica substrate having the gold coating layer on the surface was annealed at 550° C. for about 5 hr to obtain a substrate for DNA immobilization.

(Immobilization of DNA onto Substrate Surface)

In this embodiment, in order to validate the accuracy of the measurement, a known single-stranded DNA having a base length of 20 bases (SEQ ID NO: 1) shown below was used. A solution containing a predetermined concentration of the single-stranded DNA was prepared and dropped onto the gold coating layer of the substrate surface for DNA immobilization which was prepared above, and the substrate was dried by being left to stand. Thus, the single-stranded DNA was immobilized on the gold coating layer and was used as a sample for analysis.

3′-GACAATTAATAAATGGCGAA-5′ (SEQ ID NO: 1)

As the step (I), the shape information of the immobilized single-stranded DNA was obtained by using a carbon-nano-tube probe. Namely, the position information of the bases was obtained by scanning over the single-stranded DNA with the probe provided to a non-contact mode atomic force microscopy and was converted into a two-dimensional image of the shape of the single-stranded DNA.

As the standard nucleic acid-binding probe used in the step (II), the following four types of standard probes were prepared:

a standard probe A prepared by binding standard nucleic acid chain A₂₀ formed of 20 bases of adenine (A) alone to a front end of a carbon-nano-tube;

a standard probe B prepared by binding standard nucleic acid chain T₂₀ formed of 20 bases of thymine (T) alone to a front end of a carbon-nano-tube;

a standard probe C prepared by binding standard nucleic acid chain G₂₀ formed of 20 bases of guanine (G) alone to a front end of a carbon-nano-tube; and

a standard probe D prepared by binding standard nucleic acid chain C₂₀ formed of 20 bases of cytosine (C) alone to a front end of a carbon-nano-tube.

The presence or absence of interaction between the standard probe A and a base of a single-stranded DNA was measured for each base position sequentially by using the standard probe A with changing distance between the standard probe A and the base according to the shape information obtained in the step (I).

Continuously, the presence or absence of interaction between each of the standard probes B, C, and D and the base of the same single-stranded DNA was measured for each base position sequentially by the same process as above using the standard probes B, C, and D. Thus, four types of information converted into two-dimensional images with respect to the base positions of the single-stranded DNA were obtained. The images reflected the presence or absence of interaction with the standard nucleic acids A₂₀, T₂₀, G₂₀, and C₂₀ bound to the four standard probes A, B, C, and D, respectively.

Then, the shape information obtained in the step (I) and the results obtained in the step (II) by measuring the presence or absence of interaction with the four types of standard probes A to D were analyzed in detail while comparing them with each other.

In the single-stranded DNA immobilized on the substrate, it was observed that the 6th, 7th, 10th, and 14th base positions from the 3′-end of the nucleic acid chain specifically interacted with the standard probe A only. Namely, it was determined that the 6th, 7th, 10th, and 14th bases from the 3′-end of the nucleic acid chain were thymine because they exhibited the interaction which is characteristic between A-T.

Furthermore, it was observed that the 2nd, 4th, 5th, 8th, 9th, 11th to 13th, 19th, and 20th base positions from the 3′-end of the nucleic acid chain specifically interacted with the standard probe B only. Namely, it was determined that the 2nd, 4th, 5th, 8th, 9th, 11th to 13th, 19th, and 20th bases from the 3′-end of the nucleic acid chain were adenine because they exhibited the interaction which is characteristic between T-A.

It was observed that the 3rd and 17th base positions from the 3′-end of the nucleic acid chain specifically interacted with the standard probe C only. Namely, it was determined that the 3rd and 17th bases from the 3′-end of the nucleic acid chain were cytosine because they exhibited the interaction which is characteristic between G-C.

For the single-stranded DNA of interest, it was determined on the basis of the shape information obtained in the step (I) that the nucleic acid chain was composed of 20 bases. Additionally, from the above-mentioned results, it was concluded that the 1st, 15th, 16th, and 18th bases from the 3′-end of the nucleic acid chain were bases other than T, A, and C, i.e., they are guanine. Actually, it was observed that the 1st, 15th, 16th, and 18th base positions from the 3′-end of the nucleic acid chain specifically interacted with the standard probe D only. This agreed with the conclusion above.

With the analysis results, the nucleic acid chain of the single-stranded DNA immobilized on the substrate was confirmed to be composed of 20 bases, and all 20 bases from the 3′-end to the 20th position were identified. The determined nucleotide sequence agreed with the above-mentioned sequence (SEQ ID NO: 1).

### Embodiment 2

In this embodiment, in order to validate the measurement of the accuracy in a portion where one kind of base continues, a known single-stranded DNA having a base length of 20 bases (SEQ ID NO: 2) shown below was used. A solution containing a predetermined concentration (1 to 0.1 μg/ml) of the single-stranded DNA was prepared and dropped onto the gold coating layer of the substrate surface for DNA immobilization which was prepared above. Then, the substrate was dried by being left to stand. Thus, the single-stranded DNA was immobilized on the gold coating layer and was used as a sample for analysis.

3′-TTTTTTTTTTCCCCCCCCCC-5′ (SEQ ID NO: 2)

In the step (I), the shape information of the immobilized single-stranded DNA was obtained by using a carbon-nano-tube probe. Namely, the position information of the bases was obtained and observed by scanning over the single-stranded DNA with the probe provided to a non-contact mode AFM and was converted into a two-dimensional image of the shape of the single-stranded DNA.

Four standard probes A to D, which are used in the step (II) as the standard nucleic acid-binding probe, were prepared as in EMBODIMENT 1.

For the single-stranded DNA of interest, it was determined on the basis of the shape information obtained in the step (I) that the nucleic acid chain was composed of 20 bases. The presence or absence of interaction between the standard probe and a base of the single-stranded DNA was measured for each base position sequentially while changing distance between the standard probe and the base position according to the shape information. As a result, interaction which is characteristic between A-T was continuously observed at the portion of the 1st to 10th bases from the 3′-end of the nucleic acid chain. Thus, the 1st to 10th bases from the 3′-end of the nucleic acid chain were determined to be thymine. Then, similar measurement was conducted by using the standard probe C. As a result, interaction which is characteristic between G-C was continuously observed at the portion of the 11th to 20th bases from the 3′-end of the nucleic acid chain. Thus, the 11th to 20th bases from the 3′-end of the nucleic acid chain were determined to be cytosine.

From the results of the measurement, it was concluded that accuracy for determining the kinds of the 20 bases constituting the single-stranded DNA of interest was sufficiently achieved by the measurement only using the standard probe A and the standard probe C. It was determined that the 1st to 10th bases from the 3′-end of the nucleic acid chain were thymine and the 11th to 20th bases were cytosine, on the basis of the shape information obtained in the step (I) and the results of the measurement for the presence or absence of interaction with the standard probe A or C. This agreed with the above-mentioned sequence (SEQ ID NO: 2).

It was confirmed that the accuracy in the determination of the nucleotide sequence was not influenced at all by omitting the measurement using the remaining standard probe B and the standard probe D.

In this embodiment, in order to actually validate the above-mentioned point, the presence or absence of interaction with the standard probe B and the standard probe D was measured for confirmation, and no characteristic interaction between T-A and between C-G was observed. Therefore, it was actually validated that the accuracy in the determination of the nucleotide sequence was absolutely not influenced at all by omitting the measurement using the remaining standard probe B and the standard probe D.

While the present invention has been described with reference to exemplary embodiments, it is to be understood that the invention is not limited to the disclosed exemplary embodiments. The scope of the following claims is to be accorded the broadest interpretation so as to encompass all modifications, equivalent structures and functions.

This application claims the benefit of Japanese Application No. 2005-088618 filed Mar. 25, 2005, which is hereby incorporated by reference herein in its entirety.

What is claimed is:

1. A method for determining a nucleotide sequence of a single-stranded nucleic acid molecule immobilized on a substrate by using a scanning probe microscope, the method comprising the steps of:
(I) analyzing a shape of bases constituting the single-stranded nucleic acid molecule by scanning over a nucleic acid chain of the single-stranded nucleic acid molecule immobilized on the substrate with a probe provided to the scanning probe microscope;
(II) preparing a standard nucleic acid-binding probe to which a standard nucleic acid is bound and detecting the presence or absence of interaction between the bases constituting the single-stranded nucleic acid molecule and the standard nucleic acid by scanning over the nucleic acid chain using the standard nucleic acid-binding probe; and
(III) determining the nucleotide sequence of the nucleic acid chain by identifying the kinds of bases existing in the nucleic acid chain on the basis of the results obtained in the steps (I) and (II).

2. The method according to claim 1, wherein the standard nucleic acid is a single-stranded DNA formed of only one kind of base selected from adenine, thymine, guanine, and cytosine.

3. The method according to claim 1, wherein a plurality of standard nucleic acid-binding probes is utilized in the step (II), and each of said plurality of standard nucleic acid-binding probes has a respective one of a plurality of standard nucleic acids bound to it, wherein each of said plurality of standard nucleic acids is a single-stranded DNA formed of only one kind of base, and wherein one of said plurality of standard nucleic acids is formed from adenine, one of said plurality or standard nucleic acids is formed from thymine, one of said plurality of standard nucleic acids is formed from guanine, and one of said plurality of standard nucleic acids is formed from cytosine.

4. The method according to claim 1, wherein the standard nucleic acid is bound to a surface of the standard nucleic acid-binding probe at the front end of the standard nucleic acid-binding probe, and wherein the standard nucleic acid-binding probe has the same shape and is made of the same material as the probe used in the step (I).

5. The method according to claim 1, wherein the detection of the presence or absence of the interaction is conducted by detecting displacement of a cantilever provided with the standard nucleic acid-binding probe.

6. The method according to claim 1, wherein the scanning probe microscope is an atomic force microscope.

7. The method according to claim 1, wherein the scanning probe microscope is a scanning tunnel microscope.


SEQUENCE LISTING (2 sequences)

SEQ ID NO: 1 - DNA, 20 bp, Artificial Sequence
  Synthesized 20mer DNA
aagcggtaaataattaacag

SEQ ID NO: 2 - DNA, 20 bp, Artificial Sequence
  Synthesized 20mer DNA
ttttttttttcccccccccc